(19) United States
(12) Patent Application Publication
(10) Pub. No.: US 2010/0030139 A1
(43) Pub. Date: Feb. 4, 2010

(54) Anastomosis Devices and Methods Utilizing Colored Bands

(76) Inventor: Vincent G. Copa, Minnetonka, MN (US)

Correspondence Address: AMS RESEARCH CORPORATION, 10700 BREN ROAD WEST, MINNETONKA, MN, 55343, US

(21) Appl. No.: 12/510,783

(22) Filed: Jul. 28, 2009

Related U.S. Application Data
(60) Provisional application No. 61/084,682, filed on Jul. 30, 2008.

(51) Int. Cl. A61B 17/12; A61M 25/10

(52) U.S. Cl. 604/100.02; 606/153

(57) ABSTRACT

Methods and apparatus for visually distinguishing an inflation balloon from a distal tip of an anastomosis device with an unassisted eye so as to enhance patient safety during an anastomosis procedure. The anastomosis device can comprise a visual reference intermediate the inflation balloon and the distal tip to visually differentiate the distal tip from the inflation balloon. The visual reference can include a colored band to assist a medical profession in determining which portions of the anastomosis device can be grasped or otherwise handled so as to avoid damage to the inflation balloon during manipulation of the anastomosis device such as during removal of the anastomosis device from packaging or during deployment of the anastomosis device during the anastomosis procedure.

### PRIORITY CLAIM

The present application claims priority to U.S. Provisional Application Ser. No. 61/084,682, filed Jul. 30, 2008 and entitled “COLORED BANDS FOR ANASTOMOSIS DEVICE”, which is herein incorporated by reference in its entirety.

### FIELD OF THE INVENTION

The present invention relates generally to anastomosis devices and associated methods of use in reconnecting tissue. More particularly, the present application relates to an improved anastomosis device used to perform anastomosis and other related surgical procedures wherein the anastomosis device includes a visual marker distinguishing a distal tip from an inflation balloon so as to avoid handling damage during manipulation of the distal tip.

### BACKGROUND OF THE INVENTION

Anastomosis procedures are required for connecting or re-connecting certain body tissues, e.g., as part of a surgical procedure. Typically, these tissues define a body lumen such as a blood vessel, intestinal, digestive or urinary tissue that are severed and/or reconnected as part of a successful treatment. One representative example can include a radical prostatectomy procedure in which a surgeon removes all or most of a patient's prostate. The procedure generally leaves a severed urethral stump and a severed bladder neck, which must be reconnected so as to restore proper urinary functions.

A representative procedure describing the use of an anastomosis device in connecting a severed urethral stump and a severed bladder neck is described in U.S. Patent Publication No. 2005/0070938A1, which is commonly assigned to the assignee of the present application, American Medical Systems of Minnetonka, Minn. Through the use of a combination of retention features including an inflation balloon and a plurality of tissue approximating structures described as extendable tines, the urethral stump and bladder neck can be aligned and retained in contact throughout a healing period for the tissue. While the tissue of the urethral stump and bladder tissue are held together during healing, the anastomosis device also provides a drainage lumen allowing bodily fluids and other materials to pass during the healing period.

While the aforementioned anastomosis device effectively reconnects tissue during surgical procedures, it would be advantageous to improve upon present designs to enhance the functionality, reliability and safety associated with use of anastomosis devices.

### SUMMARY OF THE INVENTION

The present invention is directed to an anastomosis device including an elongate body containing an inflation lumen that extends to a balloon near the distal end of the device. The device utilizes colorant in the distal balloon to clearly delineate the distal tip of the device from the balloon. This integrated feature improves the overall safety of the device by assisting a physician identify and differentiate the portions of the device so that he or she will avoid grasping the balloon portion near the distal end of the device during manipulation. By avoiding grasping of the inflation balloon, the integrity of the inflation balloon is maintained. The colorant may be utilized in a variety of sizes and shapes based upon the way the inflation ports for the balloon are arranged. For example, inflation ports can be aligned side by side or “on top” of each other and thereby require different balloon locations. Typically, a colored band will be used to separate the tip of the device from the balloon.

In one aspect of the present disclosure, a representative embodiment of an anastomosis device can comprise a catheter body having a distal end defined by a distal tip, an inflation balloon and a colored band. The colored band is generally positioned between the inflation balloon and the distal tip so as to provide a visual reference distinguishing the inflation balloon from the distal tip. By providing a visual reference, a medical professional can successfully avoid contacting the inflation balloon during manipulation of the distal end such that damage to the inflation balloon is avoided.

In another aspect of the present disclosure, a method for enhancing patient safety during an anastomosis procedure can comprise providing an anastomosis device having a visual reference distinguishing an inflation balloon from a distal tip of the anastomosis device so as to avoid inadvertent contact with the inflation balloon. In some embodiments, the method can comprise providing a colored band intermediate the inflation balloon and the distal tip. The colored band visually distinguishes the inflation balloon from the distal tip such that handling of the anastomosis device during removal from packaging or during product deployment during an anastomosis procedure can be accomplished without contacting the inflation balloon.

The above summary of the invention is not intended to describe each illustrated embodiment or every implementation of the present invention. The Figures and the detailed description that follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a perspective view of a representative embodiment of an anastomosis device according to the prior art.

FIG. 2 is a perspective view of a distal end of a representative embodiment of an anastomosis device according to the present invention.

FIG. 3 is a partial section view illustrating use of the anastomosis device of FIG. 2 in performing a radial prostatectomy.

FIG. 3A is a partial section view illustrating use of the anastomosis device of FIG. 2 in performing a radial prostatectomy.

FIG. 4 is a perspective view of a distal end of a representative embodiment of an anastomosis including an integrated mesh in a connective sheath according to the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE DRAWINGS

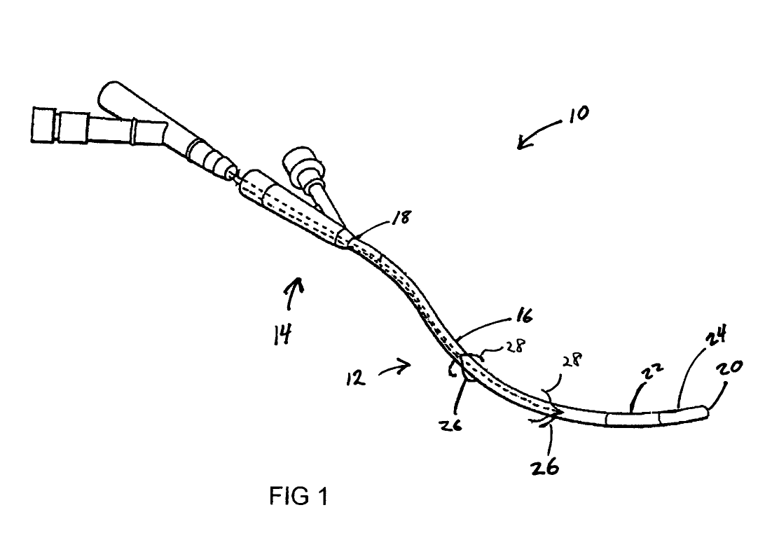

A representative embodiment of a prior art anastomosis device 10 is illustrated generally in FIG. 1. Anastomosis device 10 generally comprises a catheter portion 12 and an interface portion 14. The catheter portion 12 is generally defined by a catheter body 16 having a proximal end 18 and a distal end 20. Proximate the distal end 20, the catheter body 16 includes an inflation balloon 22, a distal tip 24, and a tissue approximating structure 26. The tissue approximating structure 26 can comprise opposed retention structures such as, for example, projecting tines 28, generally positioned along catheter body 16 between distal end 20 and proximal end 18. With the exception of the tissue approximating structure 26, the anastomosis device 10 substantially resembles the structure of a conventional Foley catheter.

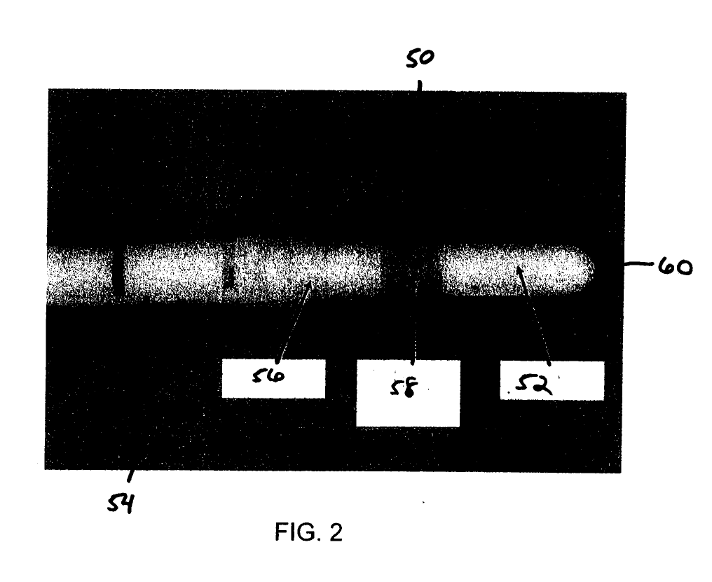

Referring now to FIG. 2, an embodiment of an anastomosis device 50 of the present invention comprises a catheter body 54 having a distal end 52, an inflation balloon 56, a colored band 58 and a distal tip 60. Colored band 58 generally includes a visible colorant that substantially contrasts with the appearance of the catheter body 54 and the inflation balloon 56. Colored band 58 visually distinguishes and separates the distal tip 60 of the anastomosis device 50 from the inflation balloon 56.

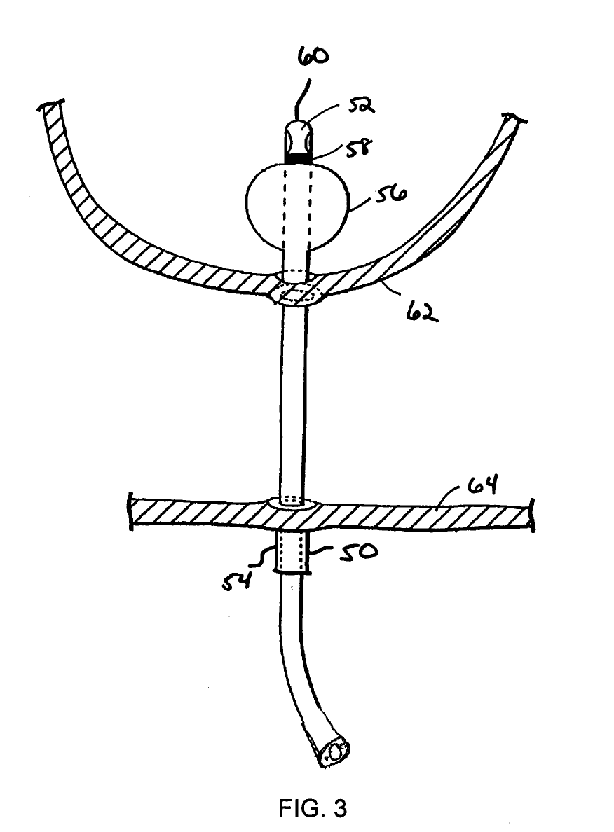

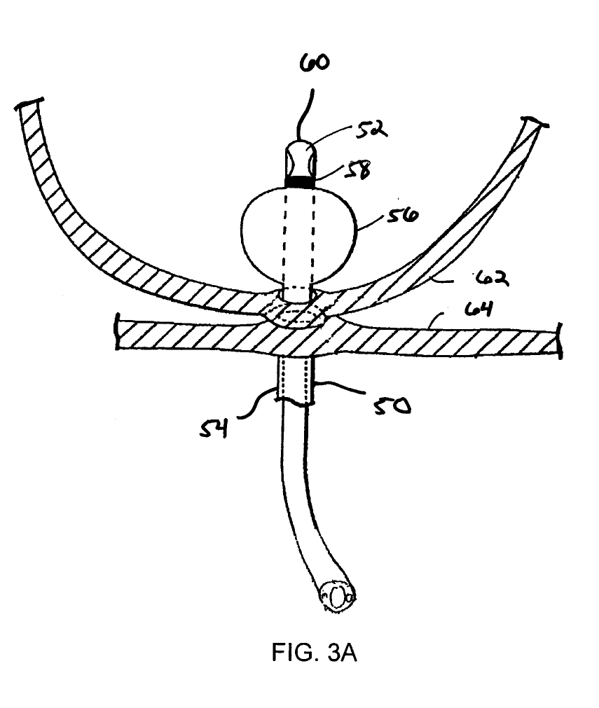

Referring now to FIGS. 3 and 3A, use of the anastomosis device 50 is illustrated with respect to reconnection of tissue following a prostatectomy. As shown in FIG. 3, bladder 62 and urethra 64 are shown in a spaced apart, unconnected disposition immediately following removal of the prostate. By gripping and retaining the bladder 62 and urethra 64 with tissue approximation structure 26, the anastomosis device 50 can approximate and reconnect bladder 62 and urethra 64 as shown in FIG. 3A. Though not essential for reconnection of the bladder 62 and urethra 64, FIGS. 3 and 3A are illustrative of the manner in which the colored band 58 visually distinguishes the inflation balloon 56 from the distal tip 60.

With the visual distinction between colored band 58 and the inflation balloon 56 as shown in FIGS. 2, 3 and 3a, a medical professional is provided a visual reference distinguishing the inflation balloon 56 from the distal tip 60. The visual reference serves to protect the inflation balloon 56 from damage as a physician manipulating and inserting the distal end 52 of the anastomosis device 50 is often required to handle the distal end 52. During handling and manipulation of the distal end 52, unintentional contact with inflation balloon 56 can lead to or otherwise contribute to failure of the inflation balloon 56. In conventional anastomosis devices including, for example, anastomosis device 10, the close proximity of the distal tip 24 and inflation balloon 22 combined with a lack of a visible reference between the inflation balloon 22 and the distal tip 24 can result in a medical professional otherwise mistaking the inflation balloon 22 for the distal tip 24 wherein damage to the inflation balloon 22 can be inflicted. If damaged, the inflation balloon 22 can be rendered inoperative such that the anastomosis device 10 is rendered unable to accomplish the anastomosis procedure.

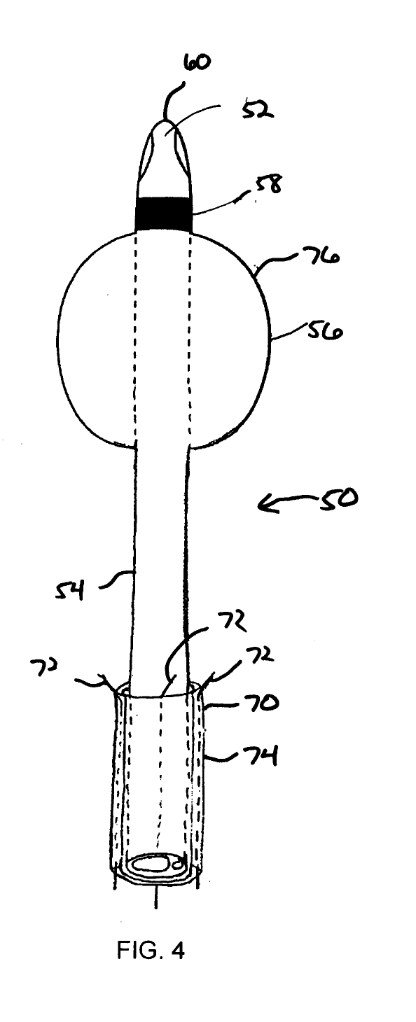

Referring now to FIG. 4, anastomosis device 50 is illustrated including tissue retention structure 70. Tissue retention structure 70 generally includes a plurality of projecting tines 72 and a sleeve 74. Through slidable manipulation of the sleeve 74, projecting tines 72 are selectively deployed or retracted during the process of device insertion, tissue retention, tissue approximation, tissue healing and device removal. As set forth in FIG. 4, inflation balloon 56 is illustrated in an inflated state 76 and projecting tine 72 are deployed which is a typical configuration after the inflation balloon 56 has been inserted into the bladder 62 and is then prepared for pulling against the perineal wall. Once again, colored band 58 of the present invention is particularly useful in allowing physicians to readily identify, distinguish and avoid contact with the inflation balloon 56 when manipulating the distal tip 60.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

1. An anastomosis device comprising:
an elongate catheter body having a proximal end and a distal end, an inflation lumen extending through the elongate catheter body from the proximal end to proximate the distal end, and a distal tip defining the distal end;
an inflation balloon proximate the distal end of the elongate catheter body; and
a colored band positioned between the inflation balloon and the distal tip to visually distinguish the distal tip from the inflation balloon, the colored band including a visible colorant such that the an unassisted eye recognizes a visual divide between the distal tip and the inflation balloon.

2. The device of claim 1, further comprising a tissue approximating structure positioned along the elongate catheter body between the distal end and the proximal end.

3. The device of claim 2, wherein the tissue approximating structure comprises opposed projecting tines.

4. The device of claim 2, wherein the tissue retention structure comprises a sleeve slidably disposed on the elongate catheter body and a plurality of projecting tines that are selectively deployable and retractable from the sleeve.

5. An anastomosis device comprising:
an elongate catheter body having a proximal end and a distal end, an inflation lumen extending through the elongate catheter body from the proximal end to proximate the distal end, and a distal tip defining the distal end;
an inflation balloon proximate the distal end of the elongate catheter body; and
means for visually distinguishing the distal tip from the inflation balloon.

6. The method of claim 5, wherein the means for visually distinguishing comprises a colored band positioned between the distal tip and the inflation balloon, the colored band possessing a color different than both the distal tip and the inflation balloon.

7. The device of claim 5, further comprising a tissue approximating structure positioned along the elongate catheter body between the distal end and the proximal end.

8. The device of claim 7, wherein the tissue approximating structure comprises opposed projecting tines.

9. The device of claim 7, wherein the tissue approximating structure comprises a sleeve slidably disposed on the elongate catheter body and a plurality of projecting tines that are selectively deployable and retractable from the sleeve.

10. A method for performing an anastomosis procedure, comprising:
providing an anastomosis device having an elongate catheter body having a proximal end, a distal end, an inflation lumen extending through the elongate catheter body between the proximal end and the distal end, and a distal tip defining the distal end, an inflation balloon proximate the distal end; and
positioning a colored band between the inflation balloon and the distal tip, the colored band providing a visual distinction to an unassisted eye such that the inflation balloon and the distal tip are visually distinguishable.

11. The method of claim 10, further comprising:
avoiding contact with the inflation balloon during manipulation of the distal tip during insertion of the distal end of the elongate catheter body within a patient.

12. A method for enhancing patient safety during an anastomosis procedure comprising:
providing an anastomosis device having an elongate catheter body defined by a proximal end, a distal end, an inflation lumen extending through the elongate catheter body between the proximal end and the distal end, and a distal tip defining the distal end, and an inflation balloon proximate the distal end; and
providing a visual reference for distinguishing a border between the inflation balloon and the distal tip using an unassisted eye.

13. The method of claim 12, wherein the visual reference is a colored band positioned between the inflation balloon and the distal tip.

14. The device of claim 16, wherein the colored band includes a visible colorant that substantially contrasts with the appearance of the distal tip and the inflation balloon.
